(19) United States
(12) Patent Application Publication
(10) Pub. No.: US 2004/0186437 A1
(43) Pub. Date: Sep. 23, 2004

(54) Content-coded medical syringe, syringe set and syringe content identification method

(76) Inventors: Claude E. Frenette, Orchard Park, NY (US); Maureen Levy, Winterhaven, FL (US); Mitchell L. Staskiewicz, Lancaster, NY (US)

Correspondence Address: Walter W. Duft, Suite 10, 10255 Main Street, Clarence, NY, 14031, US

(21) Appl. No.: 10/392,519

(22) Filed: Mar. 20, 2003

(51) Int. Cl. A61M 5/00

(52) U.S. Cl. 604/189

(57) ABSTRACT

A content-coded syringe for medical use includes a generally tubular body and a plunger slidably disposed in the body. A content code corresponding to a syringe content type to be introduced into the syringe is permanently and directly formed on the body or the plunger or both. It is selected from a set of content codes used on other syringes of like design that are adapted to receive other syringe content types. A set of such syringes with unique content codes can thus be used in a medical procedure to uniquely identify the syringe contents and thereby minimize syringe misidentification.

### BACKGROUND OF THE INVENTION

1. Field of the Invention

The present invention relates to syringes designed for medical applications, and particularly to medical syringes adapted to be pre-charged with syringe content for use during a medical procedure.

2. Description of Prior Art

By way of background, medical syringes are used to inject a wide variety of bioactive compounds in liquid form into the human body. The typical syringe is manufactured so as to be transparent or translucent, and non-colored, with the barrel being made from a plastic material such as polyethylene or polypropylene, and the plunger being made from the same material and capped with a silicone rubber tip. The needle is typically made from a metal such as stainless steel. External index markings, typically printed in black ink, are applied to the outside of the barrel to indicate volume in milliliters. No other markings are usually present, with the possible exception of a manufacturer's name or logo.

There are a variety of medical procedures, from elective surgery to emergency medical care, where several syringes are required to deliver different agents to a patient at different times during the procedure, often as sequential aliqouts. The substances to be delivered are almost exclusively clear non-colored liquids having the same visual appearance as water. These materials, however, are not as benign as water, and may cause great harm, or even death, if erroneously administered to the patient. Examples include narcotics, muscle relaxants, local anesthetics, vasopressors, vasodilators, and reversal agents.

The standard practice in preparing for a medical procedure is to remove the appropriate medications, anesthetics and other required agents (usually multiple categories are required for a single procedure) from their clearly-labeled vials and extract them into generic, unlabeled syringes of the type described above, thereby pre-charging each syringe in advance of the procedure. The pre-charged syringes are placed on a surgical tray that is situated near the medical practitioner to facilitate rapid syringe selection and deployment during the procedure.

Unfortunately, the identification of each syringe and its contents is typically left to ad-hoc placement procedures or hand-written notes scribbled on paper linens or place mats on which the syringes are situated. In the hectic environment of a surgical ward or Emergency Room, maintaining proper identification of the various syringes is difficult at best. In some instances, the wrong agent has been improperly administered due to accidental misplacement or misalignment of the syringes, occasionally with catastrophic results.

Accordingly, there is presently a need to improve the manner in which syringes and their contents are identified for medical procedure use. What is required in particular is a syringe design, coupled with a syringe content identification method, which substantially reduces the likelihood of an incorrect syringe being selected during a medical procedure, and a possibly harmful or fatal substance being administered to the patient.

### SUMMARY OF THE INVENTION

The foregoing problems are solved and an advance in the art is provided by a novel medical syringe that is content-coded to allow its content type to be quickly, precisely and continuously identified when used during a medical procedure. The content-coded syringe includes a generally tubular body having an open end adapted to receive a plunger, a partially closed end adapted to receive a needle assembly, and a central barrel portion extending between the open and partially closed ends. The barrel portion defines a fluid chamber adapted to act as a reservoir to receive and hold syringe content, and is arranged in substantially coaxial alignment with the open and partially closed ends. A plunger is slidably disposed within the syringe body, and has a tip end adapted to displace syringe content relative to the partially closed end of the body, a base end adapted to be actuated during use of the syringe, and an elongated stem extending between the tip end and the base end. A content code corresponding to a syringe content type to be introduced into the syringe is permanently and directly formed on the body, on the plunger, on a needle assembly mounted to the body, or on any combination of the above. The content code is selected from a set of content codes used on other syringes of like design that are adapted to receive other syringe content types. A plurality of such syringes with unique content codes can thus be used in a medical procedure to uniquely identify the syringe contents and thereby minimize syringe misidentification.

In exemplary embodiments of the invention, the content code can be a color code, a text code, a colored text code, or any combination of the above (numeric or alpha-numeric codes could also be used). The content code can be formed on the surface of the component that bears the code. This can be accomplished using ink or paint, a decal or adhesive label, or other permanent marking means. Using any of the foregoing application methods, the content code can be applied to all of the component's surface area, or to one or more selected portions thereof. The content code can also be disposed within the component, such as by diffusing a color code throughout the material that forms the component. It is also possible to use a machine-readable content code in conjunction with a machine that interprets the code.

The invention further contemplates a content-coded syringe set for medical use. The syringe set comprises plural syringes and accompanying needle assemblies. Each syringe and/or needle assembly bears a unique content code that visually differentiates the syringe or needle assembly from all other members of the syringe set. Thus, when the syringes are combined with the needle assemblies and arrayed in an arrangement of syringe/needle assembly pairs, with each syringe/needle assembly pair being pre-charged with syringe content and bearing one of the content codes, they can be readily differentiated from each other by virtue of the content codes.

The invention also contemplates a method for identifying syringe content in a set of syringes to be arrayed for deployment in a medical procedure. According to this method, a plurality of syringes and associated needle assemblies are selected. The syringes and/or the needle assemblies bear a unique content code that uniquely identifies a syringe content type to be introduced into each syringe/needle assembly pair. Syringe content is introduced into each syringe/needle assembly pair according to the unique content codes so that there is a correlation between the content codes and the syringe contents. This correlation can be created according to a pre-established content code correlation standard, or could be created on the fly by noting which syringe content type is introduced into each syringe/needle assembly pair. The syringe/needle assembly pairs may then be arrayed in a suitable tray arrangement for use in a medical procedure.

### BRIEF DESCRIPTION OF THE DRAWINGS

The foregoing and other features and advantages of the invention will be apparent from the following more particular description of preferred embodiments of the invention, as illustrated in the accompanying Drawings in which:

FIG. 1 is an exploded view of a content-coded syringe constructed in accordance with one embodiment of the invention using a color-coded plunger;

FIG. 1A is a cross-sectional view taken along line 1A-1A in FIG. 1;

FIG. 1B is a cross-sectional view taken along line 1B-1B in FIG. 1;

FIG. 1C is a cross-sectional view taken along line 1C-1C in FIG. 1;

FIG. 1D is a cross-sectional view taken along line 1D-1D in FIG. 1;

FIG. 1E is a cross-sectional view taken along line 1E-1E in FIG. 1,

FIG. 1F is a cross-sectional view taken along line 1F-1F in FIG. 1;

FIG. 2 is an exploded view of a content-coded syringe constructed in accordance with another embodiment of the invention using a color-coded and text-coded plunger;

FIG. 3 is an exploded view of a content-coded syringe constructed in accordance with another embodiment of the invention using a colored text-coded plunger;

FIG. 4 is an exploded view of a content-coded syringe constructed in accordance with another embodiment of the invention using a color-coded plunger and a colored text-coded body;

FIG. 5 is an exploded view of a content-coded syringe constructed in accordance with another embodiment of the invention using a color-coded plunger and text-coded body;

FIG. 6 is an exploded view of a content-coded syringe constructed in accordance with another embodiment of the invention using a color-coded body;

FIG. 7 is an exploded view of a content-coded syringe constructed in accordance with another embodiment of the invention using a color-coded and text-coded body;

FIG. 8 is an exploded view of a content-coded syringe constructed in accordance with another embodiment of the invention using a colored text-coded body;

FIG. 9 is an exploded view of a content-coded syringe constructed in accordance with another embodiment of the invention using a color-coded needle assembly;

FIG. 10 is a perspective view showing a set of content-coded syringes arrayed for used in a medical procedure;

FIG. 11 is a perspective view showing a syringe that is content-coded using a light emitting diode;

FIG. 12 is a perspective view showing a syringe that is content-coded using a radio frequency emitting identification tag that transmits a content code signal to a radio receiver unit;

FIG. 13 is a perspective view showing a syringe that is content-coded using a bar code that is read by a bar code reader unit; and

FIG. 13 is a perspective view showing a syringe that is content-coded using a magnetic strip that is read by a magnetic reader unit.

### DETAILED DESCRIPTION OF PREFERRED EMBODIMENTS

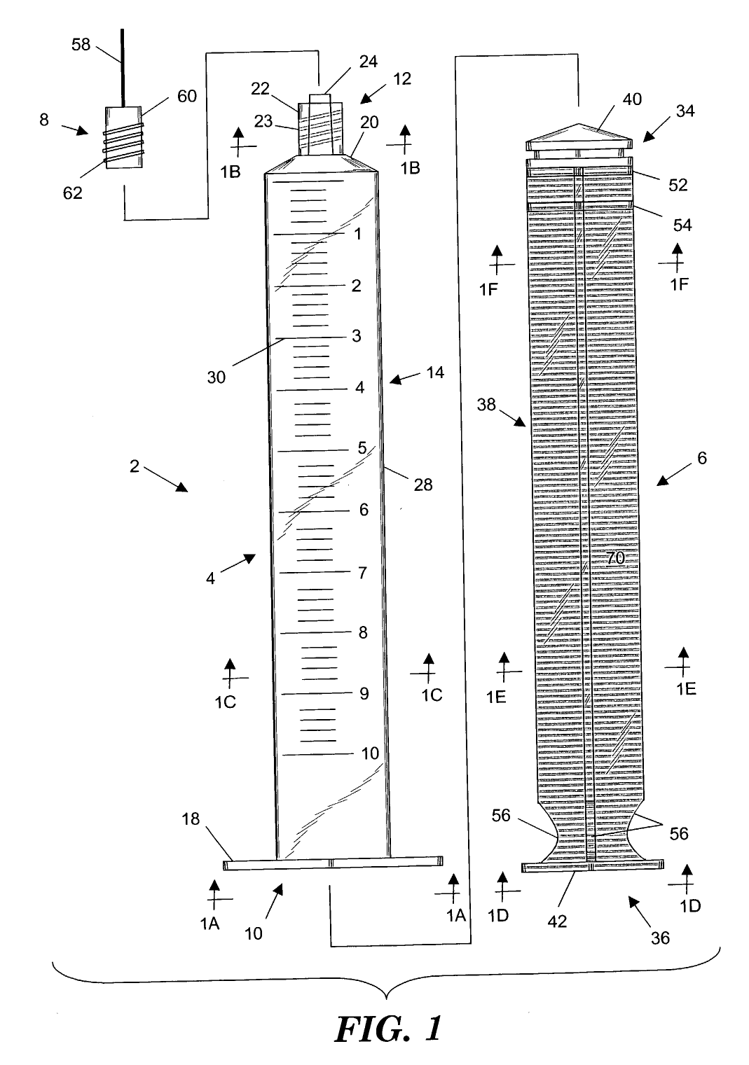

Turning now to the Drawings wherein like reference numerals signify like elements in all of the several views, FIG. 1 shows a syringe 2 that is content-coded in accordance with one exemplary embodiment of the invention. The syringe 2, whose configuration is illustrated by way of example only and not by way of limitation, is operatively composed of an elongated, generally tubular body 4 and an elongated plunger 6 that is slidably disposed within the body. A needle assembly 8, which comprises a conventional sharpened cannula and Luer lock fitting (but which could also be constructed in other ways) is adapted to mount to the body 4 prior to use of the syringe 2. Note that the needle assembly 8 is not part of the syringe 2 per se, but is considered an accessory component. Indeed, the syringe 2 would in some cases be distributed to medical facilities without the needle assembly 8 attached thereto. Instead, a removable cap member (not shown) would be provided to cover the end of the body 4 that mounts the needle assembly 8. The needle assembly 8, which could be distributed in the same packaging as the syringe 2, or in separate packaging, would be mounted to the body 4 after removing the cap member when the syringe is ready for use.

The body 4 is structurally comprised of an open end 10 that is adapted to receive the plunger 6, a partially closed end 12 that is configured to receive the needle assembly 8, and an elongated central barrel portion 14 that extends between the open and partially closed ends, and which defines a fluid chamber that acts as a reservoir for receiving and holding syringe content. These structures are characterized by various conventional features that will now be briefly described in order to provide context for the discussion of the inventive improvement to follow.

As can be seen with additional reference to FIG. 1A, the open end 10 of the body 4 is defined by an opening 16 that is circular in shape and sized to accommodate the width of the plunger 6 when the latter is introduced into the body 4. A finger press flange 18 lies in generally coplanar relationship with the opening 16, and extends outwardly from the body 4 in a direction that is transverse to the body's longitudinal dimension. The flange 18 conventionally provides a leverage point for the fingers in order to facilitate depression of the plunger 6, as well as handling and positioning of the body 4 during syringe use.

The partially closed end 12 of the body 4 is formed with a short frustoconical shoulder 20 that extends from the body's barrel portion 14 to a Luer lock tip 22. As can be seen with additional reference to FIG. 1B, the tip 22 comprises a tubular wall formed with an internal thread pattern 23 that allows it to receive and engage the needle assembly 8, which has an externally threaded Luer lock fitting. The tip 22 is also comprised of a tapered tubular nozzle 24 that is shaped as a truncated cone and formed with a central orifice 26. It will be appreciated that a Luer regular tip could be used in lieu of the Luer lock tip 22.

The barrel portion 14 of the body 4 is formed by a tubular side wall 28 that bears the usual volume markings 30, some of which are numbered. As is well known, each of the numbered markings typically represents one milliliter of syringe content. As can be seen with additional reference to FIG. 1C, the side wall 28 defines an interior fluid chamber 32 of circular cross-section. The fluid chamber 32 acts as a reservoir to receive and hold a volume of syringe content to be delivered by the syringe 2. This volume will vary depending on the position of the plunger 6, which seals the fluid chamber 32 against fluid egress out of the body's open end 10. Fluid communication into and out of the other end of the fluid chamber 32 is provided by way of the axial orifice 26 in the nozzle 24.

As is conventional, the open end 10, the partially enclosed end 12, and the barrel portion 14 of the body 4 are arranged in substantially coaxial alignment. Collectively, these structures of the body 4 can be made from a suitable thermoplastic material, such as polyethylene, polypropylene or the like, that is formed using a molding process, such as injection molding. Alternatively, glass could be used, albeit far less commonly. As is conventional, at least the barrel portion 14 of the body 4 will be relatively transparent or translucent, thereby allowing syringe content to be viewed within the fluid chamber 32, with the volume markings 30 also being visible to facilitate determination of the amount of syringe content within the syringe 2. The volume markings 30 can be conventionally applied via a suitable ink printing process to the outside of the body's tubular side wall 28.

The plunger 6 is constructed as an elongated element having a tip end 34, a base end 36, and a stem 38 extending between the tip and base ends. These structures are characterized by various conventional features that, again, will now be briefly described in order to provide context for the discussion of the inventive improvement to follow.

The tip end 34 of the plunger 6 is formed with a stopper 40, made from silicone rubber or other suitable bio-compatible material, that is adapted to seal one end of the body's fluid chamber 32. The stopper 40 also acts as a piston insofar as it is slidably positioned in fluid tight engagement with the inside of the body's tubular side wall 28. It is capable of pushing syringe content out of the fluid chamber 32, and through the orifice 26 in the nozzle 24, upon the plunger's axial advancement toward the partially closed end 12 of the body 4. Conversely, the stopper 40 is capable of pulling syringe content into the fluid chamber 32, by way of the orifice 26 in the nozzle 24, upon the plunger's axial withdrawal away from the partially closed end 12 of the body 4.

The base end 36 of the plunger 6 is adapted to be actuated during use of the syringe 2. As can be seen with additional reference to FIG. 1D, the base end 36 is formed with a thumb press flange 42 that extends transversely to the plunger's longitudinal dimension. The flange 42 conventionally provides a leverage point for the thumb or fingers in order to effect bi-directional axial movement of the plunger 6 during syringe use.

The stem 38 of the plunger 6 is an elongated member that interconnects the plunger's tip and base ends 34 and 36. As can be seen with additional reference to FIG. 1E, the stem 38 is conventionally formed with four flange elements 44, 46, 48 and 50 that share a common longitudinal edge, and which are angled at 90° to each other to form a cross-shaped configuration. These flange elements provide structural integrity to the stem 38 while reducing the amount of material required to fabricate the stem. As can be seen in FIG. 1, and with additional reference to FIG. 1F, the portion of the stem 38 that lies adjacent to the tip end. 34 of the plunger 6 is formed with a pair of closely spaced, circular disk-shaped members 52 and 54. The disk 52 provides a support base for the stopper 40. The disk 54 prevents inadvertent withdrawal of the plunger 6 from the body 4 by engaging a lip (not shown) situated on the interior of the body's tubular side wall 28 near the open end 10. As best shown in FIG. 1, the portion of the stem 38 that lies adjacent the base end 36 is formed with a cutout or depression 56 in each of the flange elements 44, 46, 48 and 50. The cutouts 56 facilitate finger engagement with the flange 42 so that the plunger 6 can be pulled out of the body 4 in order to draw syringe content into the syringe 2.

The needle assembly 8 is structurally comprised of a sharp pointed cannula 58, made from stainless steel or the like, attached to a Luer lock fitting 60 made from polyethylene, polypropylene or the like. The fitting 60 has an external thread pattern 62 that is configured to engage the internal thread pattern 23 on the syringe body's Luer lock tip 22. It will be appreciated that a non-threaded Luer regular fitting could also be used. Although not shown, the fitting 60 is formed with a central bore that is adapted to engage the outside of the tapered tubular nozzle 24 as the fitting 60 is threaded onto the body's Luer lock tip 22. This establishes a fluid tight passageway from the fluid delivery member 58, to the nozzle's orifice 26, and into the fluid chamber 32 of the body 4.

Having now described the conventional features of the syringe 2, and turning attention to the details of the inventive improvement, it will be seen in FIG. 1 that the syringe 2 is provided with an exemplary content code that corresponds to a syringe content type to be delivered by syringe. In particular, the content code of FIG. 1 is permanently formed on the plunger 6 as a color code marking 70. For purposes of illustration only, the color code marking 70 is lined to show the color blue. This color is used to signify a designated syringe content type, such as local anesthetic. The color code marking 70 will thus serve to identify all syringe content to be delivered by the syringe 2 as being of this category of bioactive agent.

The color code marking 70 is shown to extend over the entire stem 38 and base end 36 of the plunger 6. Alternatively, the color code marking 70 could be applied to just the stem 38, or to just the base end 36. It could also be applied to the stopper 40. No matter which plunger component the color code marking 70 is applied to, marking can be formed so as to be visible on all exposed surfaces of that component, or can be limited to selected areas of the component. The former implementation will ensure maximum visibility at all plunger positions. For example, if the color code marking 70 is formed over the entire stem 38, it will be relatively easy to discern even when the plunger 6 is fully disposed within the body 4 because marking will stand out through the body's transparent or translucent tubular side wall 28. The latter implementation allows variability in the way the color code marking 70 is displayed. For example, the color code marking 70 could be applied along the edges of one or more of the flanges 42, 44, 46, 48, 50, 52 and 54, or on the major planar surfaces of such flanges, or any combination of the above.

The color code marking 70 can be formed directly on the plunger 6 using a variety of application techniques. For example, the color code marking 70 could be implemented as a surface layer using ink applied by way of a printing process, paint applied by way of a painting process, or any other coating applied by way of any suitable coating technique. Alternatively, the color code marking 70 could be diffused throughout the plunger 6, as by adding it as a colorant to the thermoplastic material used to mold the plunger. A still further alternative would be apply the color code marking 70 using one or more decals or adhesive labels (e.g., color “dots”) applied to selected surface locations on the plunger 6, such as the bottom of the flange 42 at the base end 36 of the plunger.

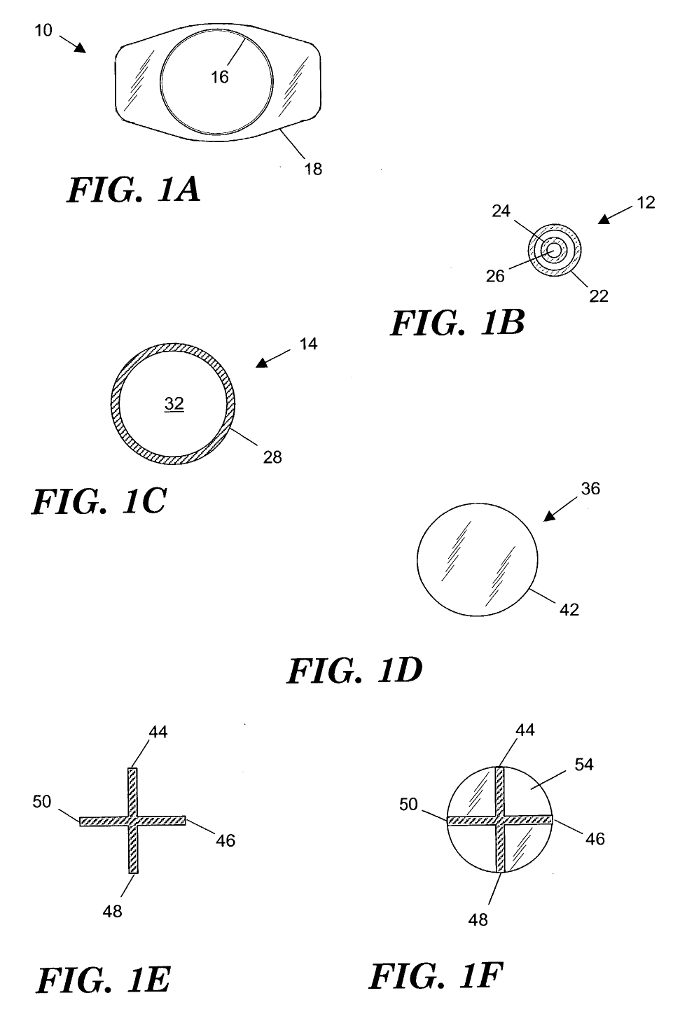

Turning now to FIGS. 2-9, several alternative embodiments of the invention will be described in order to illustrate some of the different ways that content-coded syringes may be constructed in accordance with the principles of the invention. In these alternative embodiments, the structure of each syringe is substantially similar to the syringe 2 of FIGS. 1 and 1A-1F. Accordingly, substantially similar components that perform substantially similar functions as the components of FIGS. 1 and 1A-1F are numbered identically, except that a suffix, such as “a,” “b,” “c,” etc., is used to identify the components of FIGS. 2-9.

In FIG. 2, the syringe 2a has a content code that is permanently formed on the plunger 6a as a color code marking 70a and a text code markings 72a. For purposes of illustration only, the color code marking 70a is lined for the color blue and the text code markings 72a read “Local Anesthetic.” The markings 70a and 72a will thus serve to identify all syringe content to be delivered by the syringe 2a as being of this category of bioactive agent. It will be appreciated that the color code marking 70a can be formed on the plunger 6a in the same locations as the color code marking 70 of FIG. 1, using the same application techniques. The text code markings 72a are shown to extend over a portion of the plunger stem 38a. Any other location of that is of sufficient size to carry the text could also be used. Note that any number of text markings 72a can be used. For the stem 38a, the typical number of text markings 72a would range from one marking formed on one surface of one flange to eight markings on each of the eight major surfaces of the flanges that define the stem's cross-shaped construction. Insofar as the text code markings 72a comprise text, they will typically be formed on the plunger surface using ink applied via a printing process. Alternatively, or in conjunction with ink printing, the text code markings 72a could be formed by molding surface relief indicia in the form of text into the plunger 6a during its formation. A still further alternative would be apply the text code markings 72a using one or more decals or adhesive labels bearing the text and secured to selected surface locations on the plunger 6a.

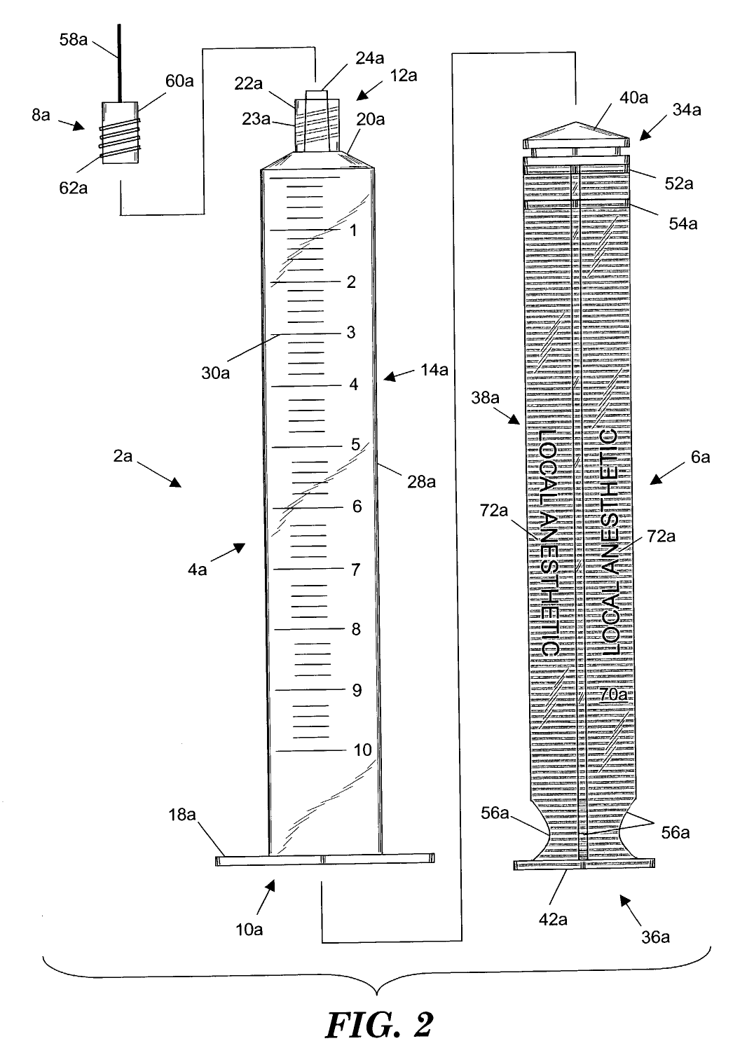

In FIG. 3, the syringe 2b has a content code that is permanently formed on the plunger 6b as color-coded text markings 72b. For purposes of illustration only, the color-coded text markings 72b are assumed to be printed in the color red and read “Narcotic.” The markings 72b will thus serve to identify all syringe content to be delivered by the syringe 2b as being of this category of bioactive agent. It will be appreciated that the color-coded text markings 72b can be formed on the plunger 6b in the same locations as the text code markings 72a of FIG. 2, using the same application techniques, but with colored ink being applied. Again, any number of color-coded text markings 72b may be formed on the plunger 6b, with a range of one to eight markings being most common given the plunger's cross-shaped stem configuration.

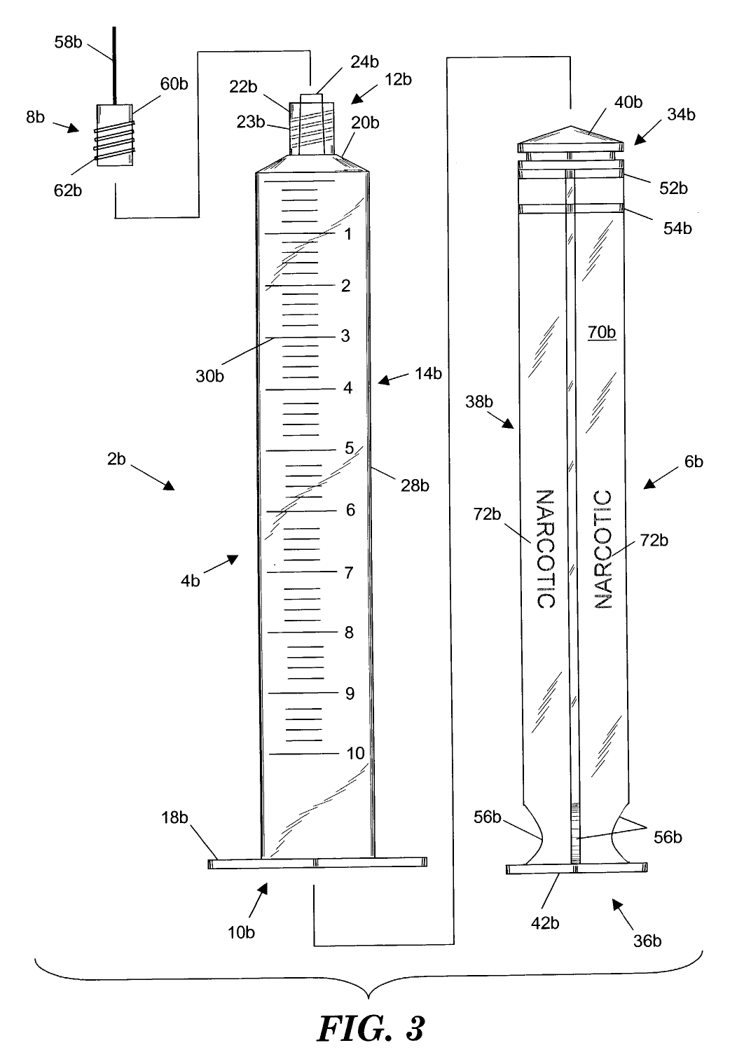

In FIG. 4, the syringe 2c has a content code that is applied to both the plunger 6c and the body 2c. The content code portion applied to the plunger 6c is permanently formed as a color code marking 70c. The content code portion applied to the body 2c is permanently formed as a corresponding color-coded text marking 74c. For purposes of illustration only, the color code marking 70c is lined for the color green and the color-coded text marking 74c is assumed to be green and reads “Reversal Agent.” Both of the markings 70c and 74c will thus serve to identify all syringe content to be delivered by the syringe 2d as being of this category of bioactive agent. It will be appreciated that the color code marking 70c can be formed on the plunger 6c in the same locations as the color code marking 70 of FIG. 1, using the same application techniques. The color-coded text marking 74c is shown to extend axially over a portion of the body's barrel portion 14c. The marking 74c could also be applied circumferentially, if space permits. Any other location of that is of sufficient size to carry the text could also be used, such as the bottom of the flange 18c, or the frustoconical shoulder 20c. If space permits, multiple color-coded text markings 74c could be applied. Insofar as the color-coded text marking 74c comprises text, it will typically be formed on the body's surface using the desired color of ink applied via a printing process. Additionally, in conjunction with ink printing, the color-coded text marking 74c could be formed by molding surface relief indicia in the form of text into the body 2c during its formation. A still further alternative would be to apply the color-coded text marking 74c using one or more decals or adhesive labels bearing the text and secured to selected surface locations on the body 2c.

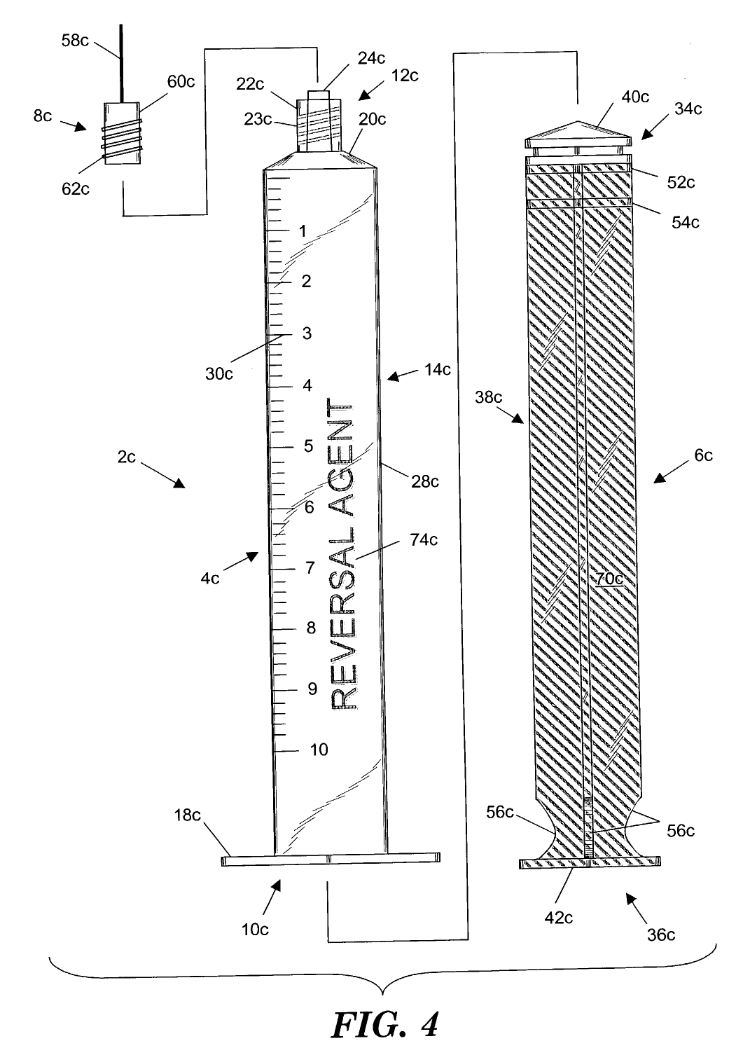

In FIG. 5, the syringe 2d has a content code that is essentially as same as the content code of FIG. 4 insofar as there is a color code marking 70d on the plunger 6d and a text code marking 74d on the body 4d. However, the text code marking 74d is not color-coded, and is printed with black lettering. For purposes of illustration only, the color code marking 70d is lined for the color yellow and the text code marking 74d reads “Relaxant.” The marking 74d will thus serve to identify all syringe content to be delivered by the syringe 2d as being of this category of bioactive agent. It will be appreciated that color code marking 70d can be formed on the plunger 6d in the same locations as the color-coded marking 70 of FIG. 1, using the same application techniques. Similarly, the text code marking 74d can be formed on the body 4d in the same locations as the color-coded text marking 74c of FIG. 4, using the same application techniques, but with black ink being used. Multiple text code markings 74d may be applied if space permits.

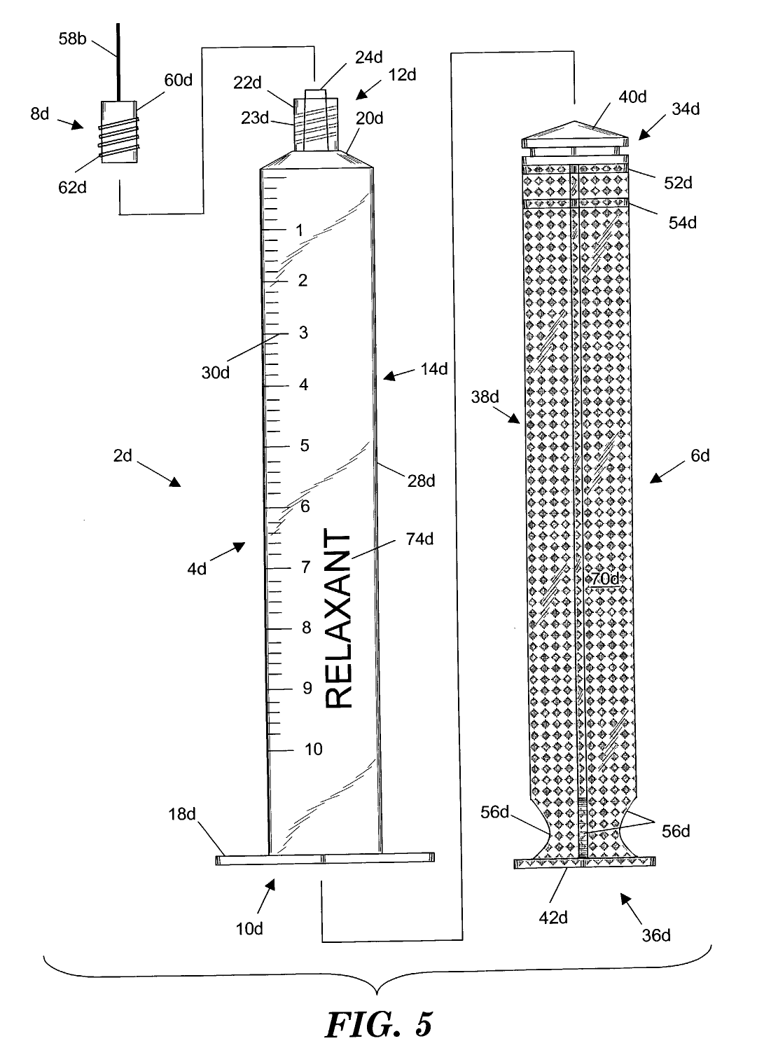

In FIG. 6, the syringe 2e has a content code that is permanently formed on the body 2d as a color code marking 76e, with no content code being applied to the plunger 2e. For purposes of illustration only, the color code marking 70 is lined to show the color orange. This color is used signify a designated syringe content type, such as vasopressor. The color code marking 76e will thus serve to identify all syringe content to be delivered by the syringe 2e as being of this category of bioactive agent.

The color code marking 76e is shown to extend over the entirety of the body 2e, including the open end 10e, the partially closed end 12e, and the elongated barrel portion 14e. Alternatively, the color code marking 76e could be applied to just the open end 10e (e.g., to the flange 18e), or to just the partially closed end 12e (e.g., to the frustoconical shoulder and/or the Luer lock fitting 22e), or to just the barrel portion 14 (e.g., to the tubular side wall 28e). No matter which body component the color code marking 76e is applied to, the marking can be formed so as to be visible on all exposed surfaces of that component, or can be limited to selected areas of the component. The former implementation will ensure maximum visibility while the latter implementation allows variability in the way the color code marking 76e is displayed. It will be appreciated that the color code marking 76e can be formed using any of the application techniques described above in connection with the color code marking 70 of FIG. 1. For example, the color code marking 76e could be implemented as a surface layer using ink applied using a printing process, paint applied using a painting process, or any other coating applied using a suitable coating technique. Alternatively, the color code marking 76e could be diffused throughout the body 4e, as by adding it as a colorant to the thermoplastic material used to mold the body. A still further alternative would be apply the color code marking 76e using one or more decals or adhesive labels (e.g., color “dots”) applied to selected surface locations on the body 4e, such as the barrel portion 14e near the flange 18e.

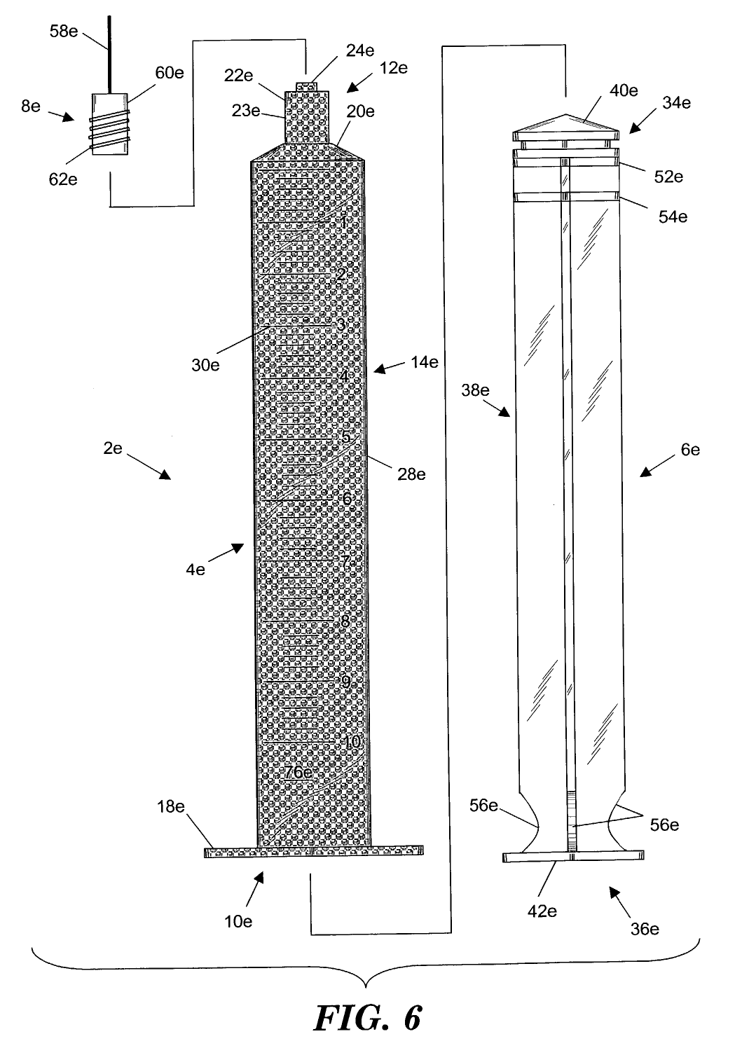

In FIG. 7, the syringe 2f has a content code that is permanently formed on the body 4f as a color code marking 76f and a text code marking 74f. For purposes of illustration only, the color code marking 76f is lined for the color orange and the text code marking 74f reads “Vasopressor.” The markings 76a and 74f will thus serve to identify all syringe content to be delivered by the syringe 2f as being of this category of bioactive agent. It will be appreciated that the color code marking 76f can be formed on the body 4f in the same locations as the color code marking 76c of FIG. 6, using the same application techniques. Similarly, the text code marking 74f can be formed on the body 4f in the same locations as the text code marking 74d of FIG. 5, using the same application techniques. Multiple text code markings 74f may be applied if space permits.

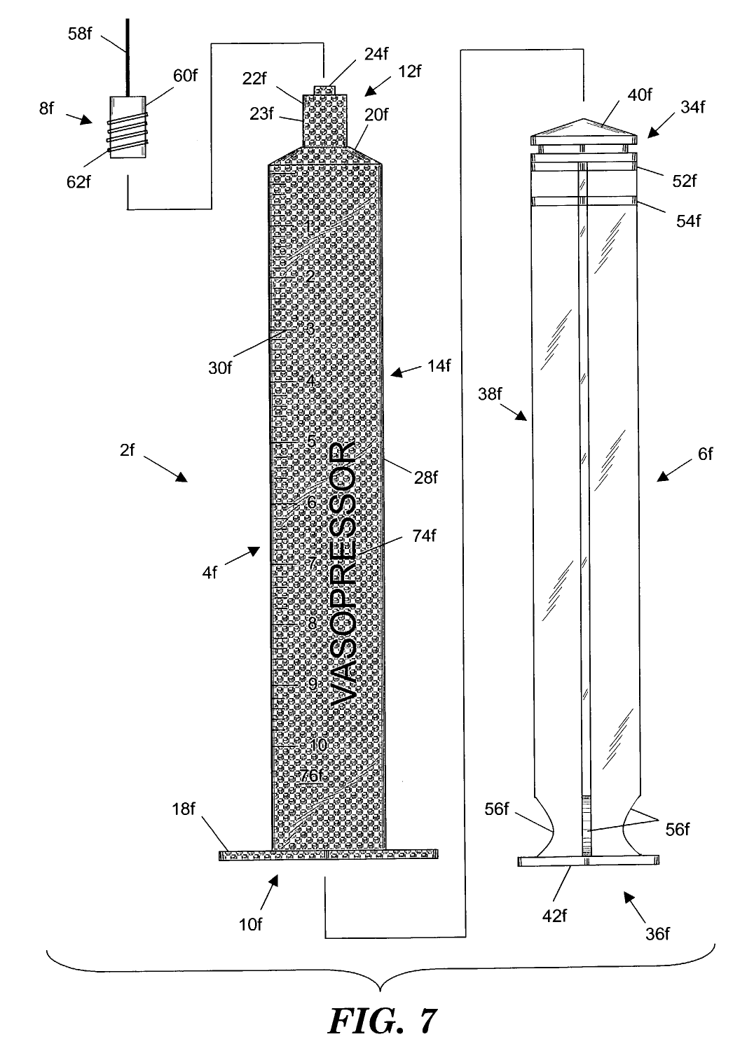

In FIG. 8, the syringe 2g has a content code that is permanently formed on the body 4g as a color-coded text marking 74g. For purposes of illustration only, the color-coded text marking 74g is assumed to be printed in the color purple and reads “Vasodilator.” The marking 74g will thus serve to identify all syringe content to be delivered by the syringe 2g as being of this category of bioactive agent. It will be appreciated that the color-coded text marking 74g can be formed on the body 4g in the same locations as the color-coded text is marking 74c of FIG. 4, using the same application techniques. Multiple color-coded text markings 74g may be applied if space permits.

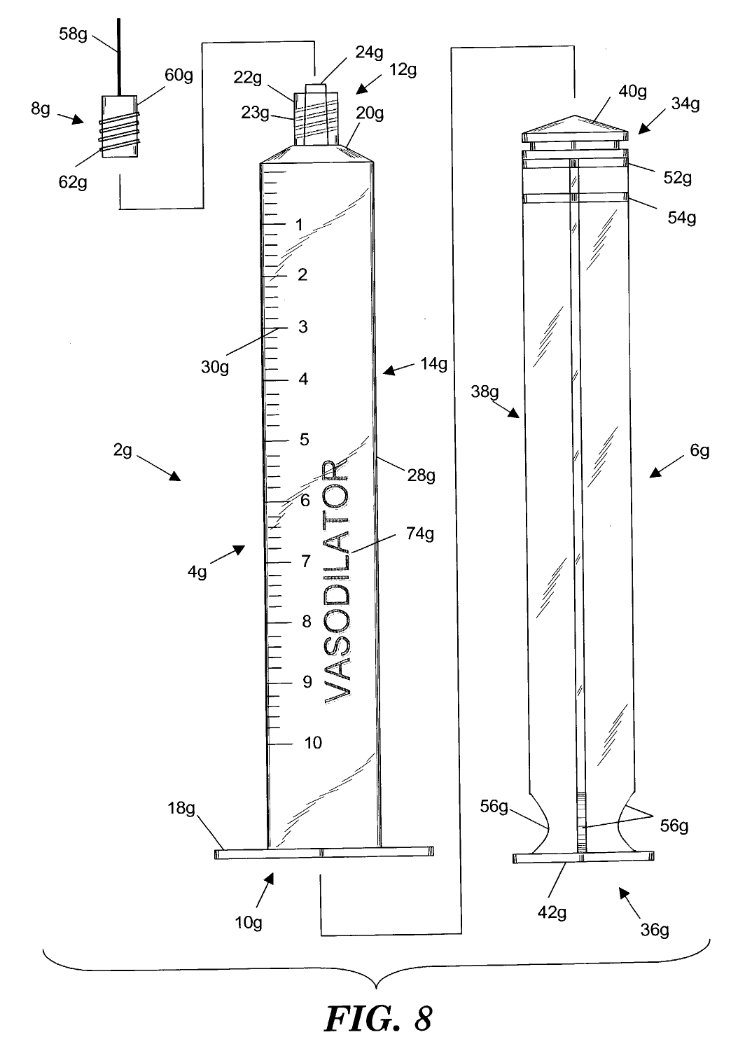

In FIG. 9, the syringe 2h has a content code that is permanently formed on the needle assembly 8h as a color code marking 78h, with no content code being applied to the body 4h or the plunger 6h. For purposes of illustration only, the color code marking 78h is lined to show the color blue. This color is used signify a designated syringe content type, such as local anesthetic. The color code marking 78h will thus serve to identify all syringe content to be delivered by the syringe 2e as being of this category of bioactive agent. The color code marking 78h is shown to extend over the entirety of the Luer lock fitting 60h of the needle assembly 8h. The color code marking 78h could also be applied to selected portions of the fitting 60h. However, visibility might be hampered given the needle assembly's small size. For that reason, even when the color code marking 78h is formed on the entirety of the fitting 60h, it may be desirable to additionally place content codes on the body 4h or the plunger 6h, both of which are substantially larger than the needle assembly 8h. It will be appreciated that the color code marking 78h can be formed using any of the application techniques described above in connection with the color code marking 70 of FIG. 1. For example, the color code marking 78h could be implemented as a surface layer using ink applied using a printing process, paint applied using a painting process, or any other coating applied using a suitable coating technique. Alternatively, the color code marking 78h could be diffused throughout the fitting 60h, as by adding it as a colorant to the thermoplastic material used to mold the base. A still further alternative would be apply the color code marking 78e using one or more decals or adhesive labels (e.g., color “dots”) applied to selected surface locations on the fitting 60h, such as above the thread pattern 62h.

It will be appreciated that the exemplary embodiments shown in FIGS. 1-9 are not exhaustive, but are intended to illustrate just some of the various ways that content codes can be applied to a syringe. Many other content code formats could also be used, depending on considerations such as manufacturing cost, readability, aesthetics, etc.

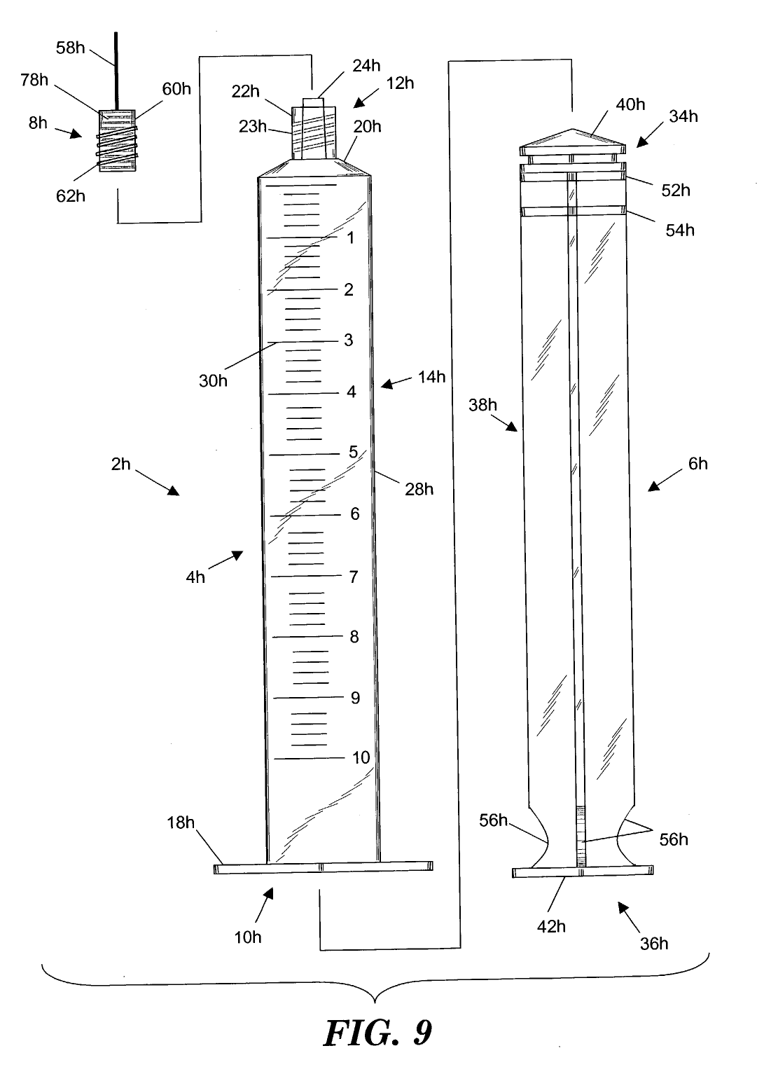

Turning now to FIG. 10, another embodiment of the invention will be described in which a syringe set 80 is formed as a collection of content-coded syringes (as described above), each of which is mounted with a needle assembly (as also previously described). There are six syringes that are respectively designated by reference numerals 80a, 80b, 80c, 80d, 80e and 80f. There are six needle assemblies that are respectively designated by reference numerals 82a, 82b, 82c, 82d, 82e and 82f. FIG. 10 also shows six content codes 84a, 84b, 84c, 84d, 84e and 84f, each of which is unique relative to the other content codes and which is permanently and directly formed on one of the syringes 80a-f. In particular, the content codes 84a-f are applied to the plungers of the syringes 80a-f as a combination of color code markings and text code markings. Other content code formats could also be used as an alternative or in addition to the foregoing. For example, color code markings and/or text code markings could be applied to the syringe bodies, to the needles assemblies 82a-f, or to both.

Each of the content codes 84a-f identifies the syringe content type to be delivered by its associated syringe 80a-f, and is selected from a set of content codes used on other syringes that are adapted to receive other syringe content types. For example, the content code 84a includes text code markings that read “Narcotic” and a color code marking that is lined for the color red. The content code 84b includes text code markings that read “Relaxant” and a color code marking that is lined for the color yellow. The content code 84c includes text code markings that read “Local Anesthetic” and a color code marking that is lined for the color blue. The content code 84d include a text code markings that read “Reversal Agent” and a color code marking that is lined for the color green. The content code 84e includes text code markings that read “Vasopressor” and a color code marking that is lined for the color orange. The content code 84f includes text code markings that read “Vasodilator” and a color code marking that is lined for the color purple.

It should be noted that the syringe content types identified by the above-described content codes 84a-f all represent categories of bioactive agents rather than individual agents. This is not intended to signify that the content codes to be utilized in accordance with the invention could never refer to individual agents. On the contrary, the concept of a syringe content type encompasses both a specific agent to be delivered by a syringe, as well as a category or family to which the specific agent belongs. For example, if a syringe is to deliver morphine, its content code could be selected to specifically identify morphine. Alternatively, the content code of the syringe containing morphine could be selected to identify the category to which morphine belongs, namely, the narcotics family. The only problem with using content codes to identify individual agents is that a relatively large number of content-coded syringes would need to be produced in order to uniquely identify each agent. On the other hand, by associating the content codes with agent categories, a much smaller number of content coded syringes are required. Moreover, most medical procedures require only one syringe agent from any given syringe agent category, thus allowing the category approach to be used in most cases because there is no need to identify specific agents. In the unlikely event that two agents in the same category do need to be separately identified for a particular medical procedure, this could be done by selecting agent-specific content codes for those syringes, even though the other syringes of the syringe set bear category-specific content codes. In other words, agent-specific and category-specific content codes may be mixed and matched within any given syringe set, depending on medical procedure requirements.

A syringe set such as that shown in FIG. 10 may be used to advantage when the syringes thereof are used in a medical procedure due to the relative ease with which each syringe and its contents may be readily and accurately identified on a continuous basis throughout the course of the procedure. Thus, according to another exemplary embodiment of the invention, an improved method is provided by which syringe content codes of the type described above are used to provide vastly improved identification of the syringe content to be delivered from a set of multiple medical procedure syringes, especially when the syringes are arrayed for deployment according to the usual side-by-side tray arrangement. Again, the content codes will typically represent categories of bioactive agents but could also represent individual agents if such specificity is required.

According to a first step of the method, a set of syringes such as that shown in FIG. 10 is selected for a medical procedure. If the syringes are not already combined with needle assemblies, then a suitable number of needle assemblies will be selected and mounted on the syringes to form syringe/needle assembly pairs. For each syringe/needle assembly pair, the syringe, the needle assembly, or both, will each have a unique content code permanently formed thereon to identify a syringe content type according to any of the content code formats discussed above. If the content code of a syringe/needle assembly pair includes text, that syringe/needle assembly pair may be used immediately to withdraw syringe content from a vial or other source of the corresponding content material. If the content code does not include text, and is based on color only, a correlation must be established that associates each color code with a particular syringe content type. In some cases, the required correlation could be established “on the fly,” with each content code being assigned to a syringe content type when the syringe or needle assembly is selected. Alternatively, the required correlation can be established in advance according to standards set by the individual medical practitioner responsible for preparing the syringe/needle assembly pairs, or more preferably, by the medical facility where the procedure is performed, or still more preferably, by a local, state or national medical board or association. The syringe set of FIG. 10 and the various individual syringe examples of FIGS. 1-9 illustrate one exemplary syringe content type/color code correlation scheme that may be used. This scheme is summarized below in Table 1:

TABLE 1
CONTENT CODE/TYPE CORRELATION SCHEME

| COLOR CODE | CONTENT TYPE |
|---|---|
| RED | NARCOTICS |
| YELLOW | RELAXANTS |
| BLUE | LOCAL ANESTHETIC |
| GREEN | REVERSAL AGENT |
| ORANGE | VASOPRESSOR |
| PURPLE | VASODILLATOR |

Assuming an appropriate content code/content type correlation has been established, or exists by default because the content codes include text, the syringe/needle assembly pairs may be pre-charged with syringe content according to the assigned content codes. The syringe/needle assembly pairs may then be arrayed in a syringe/needle assembly arrangement, on a tray or the like, for use in the medical procedure. Each syringe/needle assembly pair may now be selected for deployment as needed, with the selection being aided by the content codes that uniquely identify each syringe/needle assembly pair and their contents from all other syringe/needle assembly pairs in the arrangement. In this way, the likelihood of syringe misidentification and erroneous administration of a potentially harmful substance is greatly reduced, if not eliminated altogether.

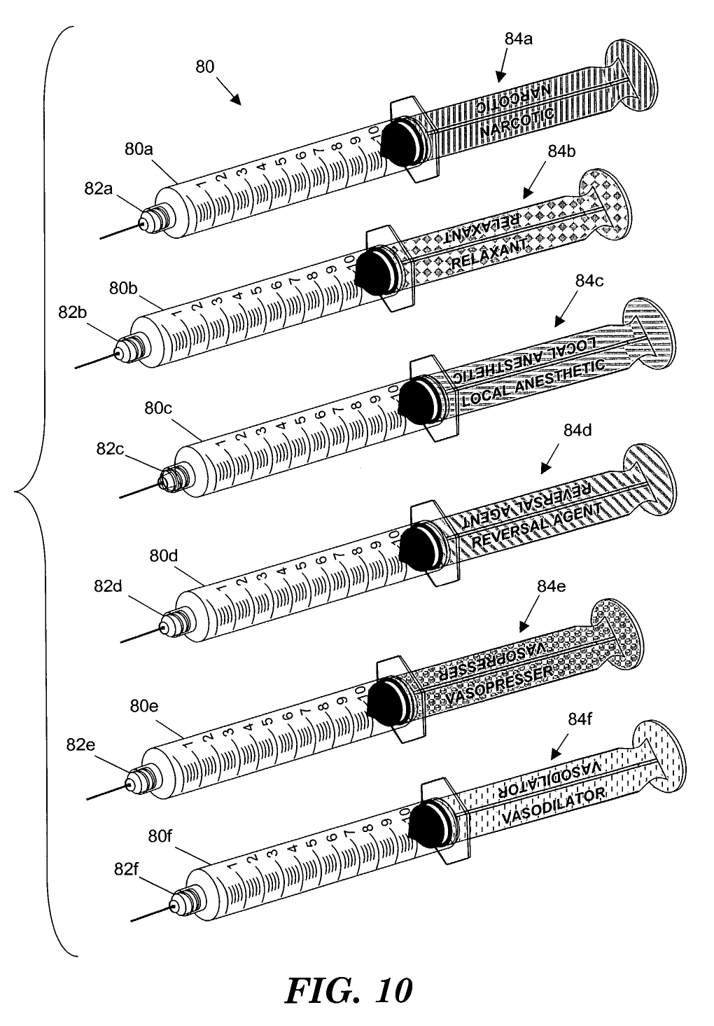

Accordingly, a content-coded syringe, together with a content-coded syringe set and syringe identification method for medical procedure use, have been disclosed. Through implementation of the invention, harmful errors and accidental deaths are more likely to be avoided because clear, precise and continuous identification of all medications, anesthetics and other agents is provided. While various embodiments of the invention have been disclosed, it should be apparent that many variations and alternative embodiments could be implemented in accordance with the teachings set forth herein. For example, instead of using a color content code formed by ink or paint or the like, FIG. 11 shows an LED (light emitting diode) 90 placed on a syringe 92. The LED 90 is selected to emit light at a characteristic wavelength to provide a color code marking.

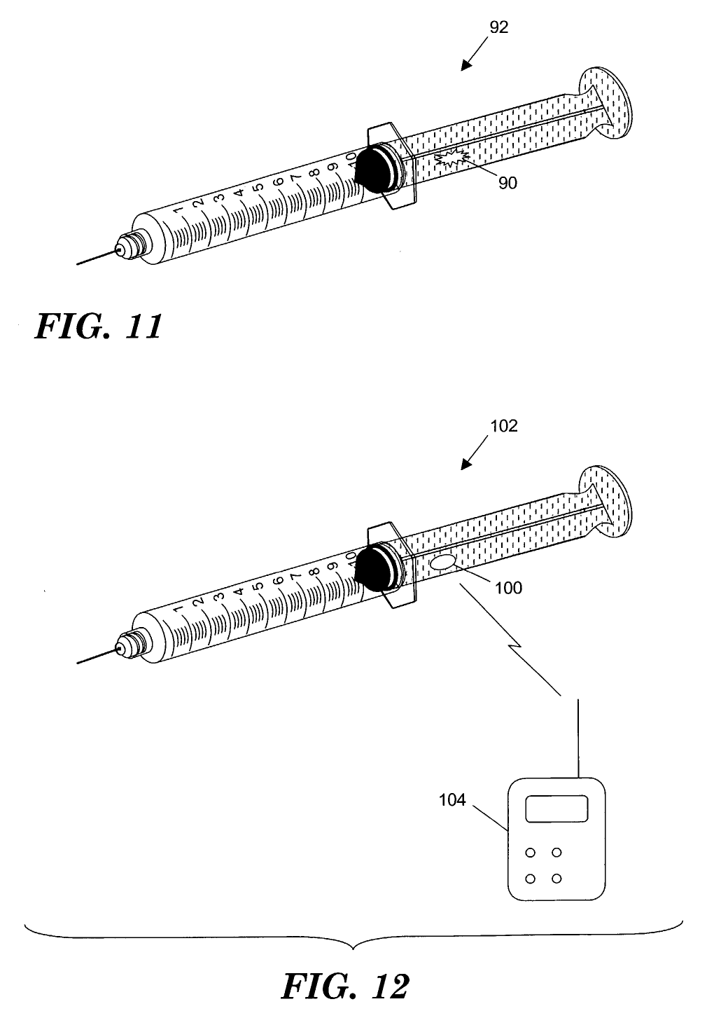

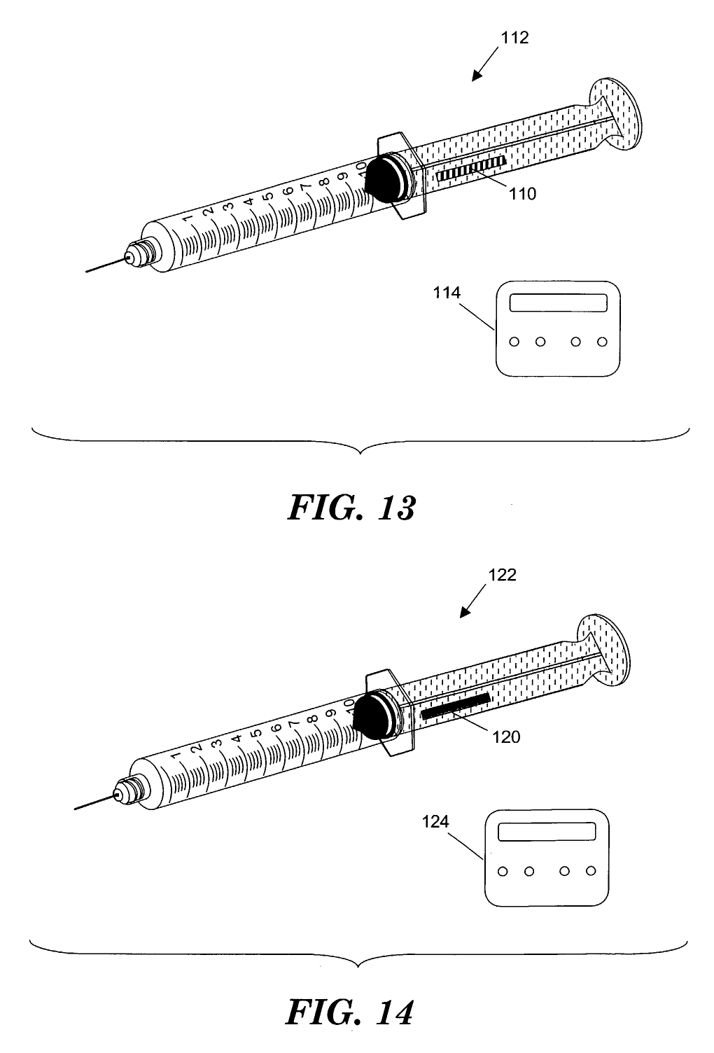

It will also be appreciated that the various content coding schemes heretofore discussed utilize human readable content codes such as color and text (human readable numeric and alpha-numeric codes could also be used). The invention could additionally be implemented using machine-readable content codes. For example, as shown in FIG. 12, an r.f. (radio frequency) identification tag 100 that transmits a low power r.f. content code signal could be placed on some portion of a syringe 102. The content code signal would be picked up by a receiver unit 104 that reads the content code and performs a verification to identify the syringe contents. In another alternative embodiment, shown in FIG. 13, a bar code 110 would be placed on some portion of a syringe 112. The bar code 112 would carry content code information and would be optically scanned by a bar code reader unit 114 that reads the content code and performs a verification to identify the syringe contents. In still another alternative embodiment, shown in FIG. 14, a magnetic strip 120 would be placed on some portion of a syringe 122. The strip would be magnetically encoded with content code information and would be sensed by a magnetic reader unit 124 that reads the content code and performs a verification to identify the syringe contents.

In view of the foregoing, it should be understood that the invention is not to be in any way limited except in accordance with the spirit of the appended claims and their equivalents.

We claim:

1. A content-coded syringe for medical use, comprising:
a generally tubular body having an open end adapted to receive a plunger, a partially closed end adapted to receive a needle assembly, and a central barrel portion extending between said open end and said partially closed end, said barrel portion defining a fluid chamber adapted to act as a reservoir to receive and hold syringe content, and said open end, said partially enclosed end and said barrel portion being arranged in substantially coaxial alignment;
a plunger slidably disposed in said syringe body, said plunger having a tip end adapted to displace syringe content relative to said partially closed end of said body, a base end adapted to be actuated during use of said syringe, and a stem extending between said tip end and said base end; and
a content code corresponding to a syringe content type to be introduced into said syringe, said content code being permanently and directly formed on said body, said plunger or both, and selected from a set of content codes used on other syringes of like design that are adapted to receive other syringe content types.

2. A content-coded syringe in accordance with claim 1, wherein said content code comprises a color code marking.

3. A content-coded syringe in accordance with claim 1, wherein said content code comprises text code marking.

4. A content-coded syringe in accordance with claim 1, wherein said content code comprises a color code marking and a text code marking.

5. A content-coded syringe in accordance with claim 1, wherein said content code comprises a color-coded text marking.

6. A content-coded syringe in accordance with claim 1, wherein said content code comprises a human-readable code.

7. A content-coded syringe in accordance with claim 1, wherein said content code comprises a machine-readable code.

8. A content-coded syringe in accordance with claim 1, wherein a content code is formed on both of said plunger and said body.

9. A content-coded syringe in accordance with claim 8, wherein said content code formed on said plunger comprises a color code marking and said content code formed on said body comprises a text code marking.

10. A content-coded syringe in accordance with claim 8, wherein said content code formed on said plunger comprises a color code marking and said content code formed on said body comprises a color-coded text marking of a color that matches said color code marking on said plunger.

11. A content-coded syringe set for medical procedure use, comprising:
a plurality of syringes, each syringe including:
a generally tubular body having an open end adapted to receive a plunger, a partially closed end adapted to receive a needle assembly, and a central barrel portion extending between said open end and said partially closed end, said barrel portion defining a fluid chamber adapted to act as a reservoir to receive and hold syringe content, and said open end, said partially enclosed end and said barrel portion being arranged in substantially coaxial alignment; and
a plunger slidably disposed in said syringe body, said plunger having a tip end adapted to displace syringe content relative to said partially closed end of said body, a base end adapted to be actuated during use of said syringe, and a stem extending between said tip end and said base end;
a plurality of needle assemblies each of which is adapted to be mounted on one of said bodies on said partially closed end thereof; and
a plurality of content codes each of which is unique and permanently and directly formed on one of said syringes or one of said needle assemblies or both to identify a syringe content type; and
whereby said syringes may be combined with said needle assemblies and arrayed in a syringe/needle assembly arrangement for use in a medical procedure, with each syringe/needle assembly being pre-charged with syringe content type and bearing one of said content codes to uniquely identify said syringe content and differentiate said syringe/needle assembly from other syringe/needle assemblies in said arrangement.

12. A content-coded syringe set in accordance with claim 11, wherein said content codes comprise color code markings.

13. A content-coded syringe set in accordance with claim 11, wherein said content codes comprise text code markings.

14. A content-coded syringe set in accordance with claim 11, wherein said content codes comprise color code markings and text code markings.

15. A content-coded syringe set in accordance with claim 11, wherein said content codes comprise color-coded text markings.

16. A content-coded syringe set in accordance with claim 11, wherein said content codes comprise human-readable codes.

17. A content-coded syringe set in accordance with claim 11, wherein said content codes comprise machine-readable codes.

18. A content-coded syringe set in accordance with claim 11, wherein said content codes are formed on said syringe bodies.

19. A content-coded syringe set in accordance with claim 11, wherein said content codes are formed on said syringe plungers.

20. A content-coded syringe set in accordance with claim 11, wherein said content codes are formed on said needle assemblies.

21. A method for identifying syringe content in a set of syringes to be arrayed for deployment in a medical procedure, comprising:
selecting a plurality of syringes, each syringe including:
a generally tubular body having an open end adapted to receive a plunger, a partially closed end adapted to receive a needle assembly, and a central barrel portion extending between said open end and said partially closed end, said barrel portion defining a fluid chamber adapted to act as a reservoir to receive and hold syringe content, and said open end, said partially enclosed end and said barrel portion being arranged in substantially coaxial alignment; and
a plunger slidably disposed in said syringe body, said plunger having a tip end adapted to displace syringe content relative to said partially closed end of said body, a base end adapted to be actuated during use of said syringe, and a stem extending between said tip end and said base end;
selecting a plurality of needle assemblies each of which is adapted to be mounted on one of said bodies on said partially closed end thereof;
said syringes or said needle assemblies or both each having a unique content code permanently and directly formed thereon to identify a syringe content type;
mounting said needle assemblies on said syringes to form syringe/needle assembly pairs, as necessary;
introducing syringe content types into said syringes according to said unique content codes such that a correlation exists between said content codes and said syringe content types; and
arraying said syringe/needle assembly pairs in a syringe/needle assembly pair arrangement for use in a medical procedure;
whereby each syringe/needle assembly pair is pre-charged with syringe content and bears one of said content codes to uniquely identify said syringe content and differentiate said syringe/needle assembly pair from other syringe/needle assembly pairs in said arrangement.

22. A method in accordance with claim 21, wherein said content codes comprise color code markings.

23. A method in accordance with claim 21, wherein said content codes comprise text code markings.

24. A method in accordance with claim 21, wherein said content codes comprise color code markings and text code markings.

25. A method in accordance with claim 21, wherein said content codes comprise colored text code markings.

26. A method in accordance with claim 21, wherein said content codes comprise human-readable codes.

27. A method in accordance with claim 21, wherein said content codes comprise machine-readable codes.

28. A method in accordance with claim 21, wherein said content codes are formed on said syringe bodies.

29. A method in accordance with claim 21, wherein said content codes are formed on said syringe plungers.

30. A content-coded syringe set in accordance with claim 21, wherein said content codes are formed on said needle assemblies.

31. A content-coded syringe and needle assembly for medical use, comprising:
a syringe having generally tubular body having an open end adapted to receive a plunger, a partially closed end adapted to receive a needle assembly, and a central barrel portion extending between said open end and said partially closed end, said barrel portion defining a fluid chamber adapted to act as a reservoir to receive and hold syringe content, and said open end, said partially enclosed end and said barrel portion being arranged in substantially coaxial alignment;
a plunger slidably disposed in said syringe body, said plunger having a tip end adapted to displace syringe content relative to said partially closed end of said body, a base end adapted to be actuated during use of said syringe, and a stem extending between said tip end and said base end;
a needle assembly adapted to mount on said partially closed end of said syringe body; and
a content code corresponding to a syringe content type to be introduced into said syringe, said content code being permanently and directly formed on one or more of said body, said plunger or said needle assembly, and selected from a set of content codes used on other syringe/needle assemblies of like design that are adapted to receive other syringe content types.
